(19) United States
(12) Patent Application Publication
(10) Pub. No.: US 2021/0369251 A1
(43) Pub. Date: Dec. 2, 2021

(54) APPARATUS FOR COLLECTING AND PACKAGING FECES

(71) Applicants: COOPERATION FOUNDATION JEONBUK NATIONAL UNIVERSITY, Jeonju-si (KR); Jeonbuk National University Hospital, Jeonju-si (KR)

(72) Inventors: Min Gul KIM, Jeonju-si (KR); Song Hee HAN, Jeonju-si (KR); Seol Ju MOON, Jeonju-si (KR)

(21) Appl. No.: 17/332,138

(22) Filed: May 27, 2021

(30) Foreign Application Priority Data
May 27, 2020 (KR) 10-2020-0063744

(51) Int. Cl. A61B 10/00; B65B 51/10; B65B 57/10; B65B 43/16; B65B 43/46; A61B 90/94

(52) U.S. Cl. CPC A61B 10/0038; B65B 51/10; A61B 90/94; B65B 43/16; B65B 43/465; B65B 57/10

(57) ABSTRACT

This application relates to configured to a feces collecting and packaging apparatus for performing automated operations of collecting and packaging the entire excrements. In one aspect, a bag supply machine of the apparatus dispenses a plastic bag with an upper opening directed into an inner space of a main body when a subject is on a seat of the main body. Following excretion, a sealing machine of the apparatus closes the upper opening of the plastic bag when the entire stools are completely contained. A collection machine of the apparatus secures the sealed plastic bag from the inner space of the main body.

### CROSS-REFERENCE TO RELATED APPLICATION

The present application claims priority to Korean Patent Application No. KR 10-2020-0063744 filed on May 27, 2020, in the Korean Intellectual Property Office, which is incorporated herein in its entirety by reference.

### TECHNICAL FIELD

The present disclosure relates to a feces collecting and packaging apparatus for performing automated operations of collecting and packaging the entire excrements

### BACKGROUND

With the increasing awareness of health, biomedical research is garnering attention and support; the sheer number of clinical research has grown remarkably over the recent years and attempts are being made to integrate the findings into routine clinical practice. The studies conducted have recognized the importance of systemic links among organ systems in our body—digestive, cardiovascular, immune, and neurologic, etc.—that they are interconnected and dependent upon each other for proper functioning. The aforementioned studies have also found that the factors promoting health and wellness are closely related to metabolism and immune system. The scope of biomedical research has further expanded to include the factors concerned with the effects of metabolism and immune system on human health. To be specific, finding a link between intestinal microbial organism and physical wellness.

As a part of intestinal microbial analysis, feces from test subjects (also referred to as examinees, study participants, or users) are collected to examine health status. The process of collecting feces is an arduous task: typically, feces are collected manually by hand, but such procedure possesses substantial risk of cross contamination and extra precautionary measures need to be taken during the process of collection. In addition, displeasing elements of manual fecal collection, related to visual, psychological, and odor-related components, may deter researchers and staffs involved in collection process.

Aside from issues of sanitation and difficulties associated with collecting samples, quality of fecal specimens is also affected. Manual collection by hand introduces inconsistency as the process is heavily dependent upon the researchers and staffs' experience and skills. Such lack of calibration hampers integrity of data generated as it complicates deriving meaningful comparisons of clinical outcomes. Moreover, for the analysis of intestinal microbes (metagenomics), it is necessary to collect the entire feces while it may not even be feasible to collect the entire feces by hand. Given the context, developing an alternative method to the present feces collection process is imperative to make feasible hygienic and quality biomedical research and applications at the clinical practice level.

### SUMMARY

To address aforementioned limitations of fecal matter collection in biomedical research, the present disclosure is to provide a feces collecting and packaging apparatus for performing automated operations of collecting and packaging the entire excrements.

According to embodiments of the disclosure, an apparatus for collecting and packaging feces may include a main body including a seat, a body block supporting the seat and having an inner space into which examinee's feces are discharged, and a transfer machine, installed at an outlet of the inner space opposite to an inlet connected to the seat, transferring a plastic bag containing entire feces from the inner space; a bag supply machine dispensing the plastic bag having an upper opening into the inner space; a sealing machine installed under the transfer machine and sealing the upper opening of the plastic bag containing the entire feces; a collection machine securing the sealed plastic bag from the inner space; and a controller directing the bag supply machine to supply the plastic bag into the inner space when the examinee is seated on the seat, directing the sealing machine to seal the upper opening of the plastic bag when the entire feces are completely contained, and controlling the collection machine to secure the sealed plastic bag from the inner space.

The controller may instruct the transfer machine to close the outlet of the inner space while the plastic bag is supplied into the inner space and then the feces are contained in the plastic bag. When the entire feces are completely contained in the plastic bag, the controller may instruct the transfer machine to move the plastic bag under its own weight from the inner space to the sealing machine.

The bag supply machine may include a bag loading box loading a plurality of plastic bags therein, and a gripper, movably connected to the inner space, gripping the plastic bag one by one from the bag loading box, transferring the gripped plastic bag into the inner space, and expanding or closing the upper opening of the plastic bag.

When the entire feces are completely contained in the plastic bag, the controller may control the gripper to release a state of fixing the plastic bag such that the plastic bag containing the entire feces moves under its own weight from the inner space to the sealing machine.

The sealing machine may include a sealing chamber connected to the inner space via the transfer machine, and a sealer automatically sealing the upper opening of the plastic bag moved into the sealing chamber.

The collection machine may include a collection box installed adjacent to the sealing chamber, and a transfer member transferring the sealed plastic bag from the sealing chamber to the collection box under control of the controller.

The apparatus may further include a labeling machine installed in one of the sealing chamber, the inner space, or the bag supply machine, and attaching a label with examinee's feces collection information to the plastic bag under control of the controller.

According to embodiments of the disclosure, an apparatus for collecting and packaging feces may include a main body including a seat, a body block supporting the seat and having an inner space into which examinee's feces are discharged, and a transfer machine, installed at an outlet of the inner space opposite to an inlet connected to the seat, transferring a plastic bag containing entire feces from the inner space; a bag supply machine dispensing the plastic bag having an upper opening into the inner space; a sealing machine installed in the inner space and sealing the upper opening of the plastic bag containing the entire feces; a collection machine installed under the transfer machine and collecting the sealed plastic bag from the inner space; and a controller directing the bag supply machine to supply the plastic bag into the inner space when the examinee is seated, directing the sealing machine to seal the upper opening of the plastic bag when the entire feces are completely contained, and controlling the collection machine to collect the sealed plastic bag from the inner space.

The controller may instruct the transfer machine to close the outlet of the inner space while the plastic bag is supplied into the inner space and then the feces are contained in the plastic bag. When the entire feces are completely contained in the plastic bag, the controller may instruct the transfer machine to transfer the sealed plastic bag under its own weight from the inner space to the collection machine.

The apparatus may further include a labeling machine installed in one of the sealing chamber, the inner space, or the bag supply machine, and attaching a label with examinee's feces collection information to the plastic bag under control of the controller.

According to the present disclosure, the feces collecting and packaging apparatus does not require human involvement by automating the whole processes of collecting and packaging excrements. Therefore, it is possible to reduce the risk of cross contamination that may occur in the process of collecting the entire feces. Further, it is expected that the speed at which fecal samples are collected can be increased, and also visual, psychological, and odor-related discomfort reduced.

### BRIEF DESCRIPTION OF THE DRAWINGS

FIG. 1 is a block diagram illustrating a feces collecting and packaging apparatus according to the present disclosure.

FIG. 2 is a block diagram illustrating a detailed configuration of the feces collecting and packaging apparatus shown in FIG. 1.

FIG. 3 is a diagram illustrating a feces collecting and packaging apparatus according to a first embodiment of the present disclosure.

FIGS. 4 and 5 are diagrams illustrating a process of the apparatus shown in FIG. 3 in which the bag supply machine dispenses a plastic bag into the inner space of the main body.

FIG. 6 is a diagram illustrating a feces collecting and packaging apparatus according to a second embodiment of the present disclosure.

FIGS. 7 and 8 are diagrams illustrating a process performed by the apparatus shown in FIG. 6 in which the bag supply machine dispenses a plastic bag into the inner space of the main body.

FIGS. 9 and 10 are diagrams illustrating a process of the apparatus shown in FIG. 6 in which the sealing machine seals the plastic bag and the labeling machine attaches a label to the plastic bag.

### DETAILED DESCRIPTION

Hereinafter, embodiments of the present disclosure will be described in detail with reference to the accompanying drawings. The present disclosure may, however, be embodied in many different forms and should not be construed as being limited to the embodiments set forth herein. Rather, these embodiments are provided so that the disclosure will be thorough and complete and will fully convey the scope of the disclosure to those skilled in the art.

In the following description of embodiments, techniques that are well known in the art and not directly related to the present disclosure are not described. This is to clearly convey the subject matter of the present disclosure by omitting an unnecessary explanation. For the same reason, some elements in the drawings are exaggerated, omitted, or schematically illustrated. Also, the size of each element does not entirely reflect the actual size. In the disclosure, the same or corresponding elements are denoted by the same reference numerals.

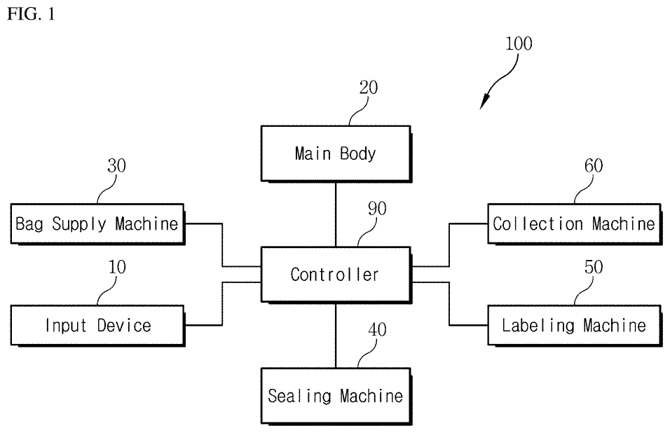

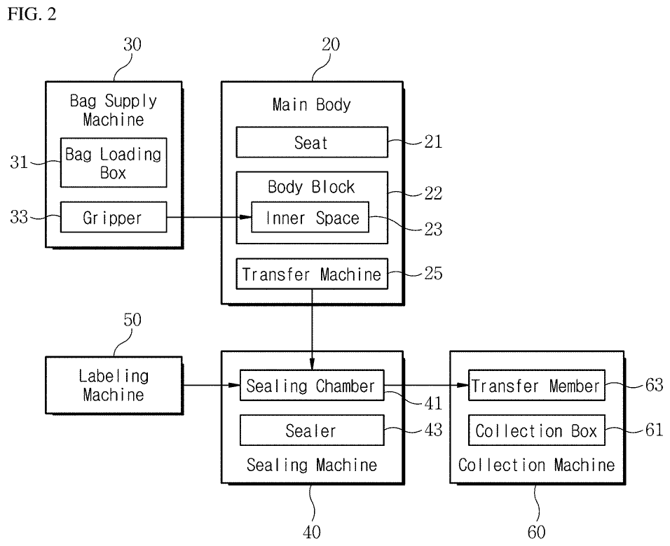

FIG. 1 is a block diagram illustrating a feces collecting and packaging apparatus according to the present disclosure. In addition, FIG. 2 is a block diagram illustrating a detailed configuration of the feces collecting and packaging apparatus shown in FIG. 1.

Referring to FIGS. 1 and 2, the feces collecting and packaging apparatus 100 according to the present disclosure is an automated device that automatically performs a series of operations of collecting entire feces from an examinee (also referred to as a test subject, a study participant, a user, etc.) and packaging the collected feces.

The feces collecting and packaging apparatus 100 includes a main body 20, a bag supply machine 30, a sealing machine 40, a collection machine 60, and a controller 90. The main body 20 includes a seat 21 and a body block 22. The seat 21 allows the examinee to be seated. The body block 22 supports the seat 21 and has an inner space 23 into which examinee's feces are discharged. The bag supply machine 30 dispenses a plastic bag having an upper opening into the inner space 23 of the body block 22. The sealing machine 40 seals the upper opening of the plastic bag containing the entire feces. The collection machine 60 secures the sealed plastic bag from the inner space 23. The controller 90 directs the bag supply machine 30 to supply the plastic bag into the inner space 23 when the examinee is seated on the seat 21. Also, the controller 90 directs the sealing machine 40 to seal the upper opening of the plastic bag when the entire feces are completely contained. Also, the controller 90 instructs the collection machine 60 to collect the sealed plastic bag from the inner space 23.

In addition, the feces collecting and packaging apparatus 100 may further include an input device 10 and a labeling machine 50.

The input device 10 receives an input from a user (i.e., examinee) for commanding the feces collecting and packaging apparatus 100 and transmits the user's input to the controller 90. For example, the user may start a feces collecting and packaging process by selecting a start button through the input device 10. Also, the user may input feces collection information through the input device 10. The feces collection information may include identification information of the examinee. The identification information may include examinee's birthdate, gender, phone number, and the like. The input device 10 may be implemented as a jog dial, a plurality of buttons, a keypad, a touchpad, a touch screen, or any combination thereof.

Alternatively, the identification information may also be in a form of identification code provided by investigator and staff to identify the examinee. For example, the identification code may be a radio frequency identification (RFID) tag, a barcode, a quick response (QR) code, or the like. In this case, the input device 10 may include a reader that can read the identification code.

The input device 10 may further include a communication interface for communicating with a communication terminal used by the examiner or the examinee. The input device 10 may receive examinee's feces collection information through the communication interface. The communication terminal may be an examiner's or examinee's portable device or a dedicated terminal provided by an inspection institution.

The main body 20 includes the seat 21 and the body block 22 as mentioned above, and may further include a transfer machine 25.

The body block 22 may be implemented in the form of a toilet bowl, and the inner space 23 is formed therein. An upper portion of the inner space 23 is used as an inlet through which feces enter, and a lower portion is used as an outlet through which a plastic bag containing the entire feces is discharged. A rear portion of the inner space 23 may be connected to the bag supply machine 30.

The seat 21 is installed around the inlet of the inner space 23. The seat 21 may be installed similar to that installed on a general toilet seat.

The transfer machine 25 is installed at the outlet of the inner space 23. The transfer machine 25 may be movably connected to one of the sealing machine 40 or the collection machine 60 located below. The transfer machine 25 blocks the outlet of the inner space 23 at normal times. When a plastic bag containing the entire feces is loaded, the transfer machine 25 moves downward toward the sealing machine 40 or the collection machine 60.

The transfer machine 25 not only moves up and down, but may also transfer the sealed plastic bag in a horizontal direction as needed. That is, the transfer machine 25 performs vertical movement and may operate like a belt conveyor that moves in a horizontal direction.

The bag supply machine 30 is installed adjacent to the main body 20 and dispenses a plastic bag that can receive feces into the inner space 23 of the main body 20. The bag supply machine 30 includes a bag loading box 31 and a gripper 33. The bag loading box 31 loads a plurality of plastic bags therein, and the gripper 33 is movably connected to the inner space 23. The gripper 33 grips a plastic bag one by one from the bag loading box 31, and transfers the gripped plastic bag into the inner space 23. After transferring the plastic bag, the gripper 33 can expand or close the opening of the plastic bag.

The gripper 33 may transfer the plastic bag into the inner space 23 through the rear of the inner space 23. A pair of grippers 33 may be located on both sides of the plastic bag. The gripper 33 vacuum-adsorbs the upper end of the plastic bag having the upper opening, moves into the inner space 23, and expands the upper opening of the plastic bag so that feces can be inserted through the upper opening of the plastic bag. In addition, the gripper 33 may close the upper opening of the plastic bag when the entire feces are completely enclosed.

The gripper 33 may release the vacuum adsorption onto the plastic bag such that the plastic bag containing the entire feces will be transferred to the sealing machine 40 or the collection machine 60.

The sealing machine 40 may include a sealing chamber 41 connected to the inner space 23 via the transfer machine 25, and a sealer 43 that automatically seals the upper opening of the plastic bag moved into the sealing chamber 41.

The sealing chamber 41 may be disposed under the inner space 23. In this case, the transfer machine 25 loading the plastic bag with the feces contained is lowered into the sealing chamber 41 by its own weight.

The sealer 43 may be a thermo-compression type sealer 43 that seals the upper end of the plastic bag having the upper opening in a thermo-compression method. The sealer 43 may remove air in the plastic bag before sealing the upper end of the plastic bag.

Alternatively, the sealing machine 40 may be installed in the inner space 23, and the collection machine 60 may be installed under the transfer machine 25. In this case, the transfer machine 25 closes the outlet of the inner space 23 while the plastic bag is supplied into the inner space 23 and then the feces are contained in the plastic bag. When the entire feces are completely enclosed in the plastic bag, the sealing machine 40 seals the upper opening of the plastic bag disposed in the inner space 23. Then, the gripper 33 releases a state of fixing (e.g., vacuum-adsorbing) the plastic bag, and the transfer machine 25 transfers the sealed plastic bag by its own weight from the inner space 23 to the collection machine 60.

The labeling machine 50 is installed in the sealing chamber 41, and attaches a label with examinee's feces collection information to the sealed plastic bag under the control of the controller 90. For example, the label may be an adhesive sticker containing the feces collection information.

Although a case where the labeling machine 50 is installed to be connected to the sealing machine 40 is described above, the present disclosure is not limited thereto. In another example, the labeling machine 50 may be installed in the inner space 23 of the main body 20. In still another example, the labeling machine 50 may be installed in the bag supply machine 30. In this case, after the gripper 33 adsorbs the plastic bag and before the gripper 33 supplies the plastic bag into the inner space 23, the labeling machine 50 may attach the label with examinee's feces collection information to the plastic bag.

The collection machine 60 includes a collection box 61 and may further include a transfer member 63. The collection box 61 is installed adjacent to the sealing chamber 41. The transfer member 63 transfers the sealed plastic bag from the sealing chamber 41 to the collection box 61 under the control of the controller 90.

The transfer member 63 may be a pusher or a belt conveyor. In the former case, the pusher pushes the sealed plastic bag residing in the sealing chamber 41 to the collection box 61. In the latter case, one side of the belt conveyor is located adjacent to the sealing chamber 41, and the other side is connected to the collection box 61. The sealed plastic bag loaded on the transfer machine 25 in the sealing chamber 41 is transferred onto the belt conveyor by the linear rotational motion (i.e., based on a closed loop of belt) of the transfer machine 25. Then the sealed plastic bag is moved to the other side of the belt conveyor by the operation of the belt conveyor and is collected into the collection box 61.

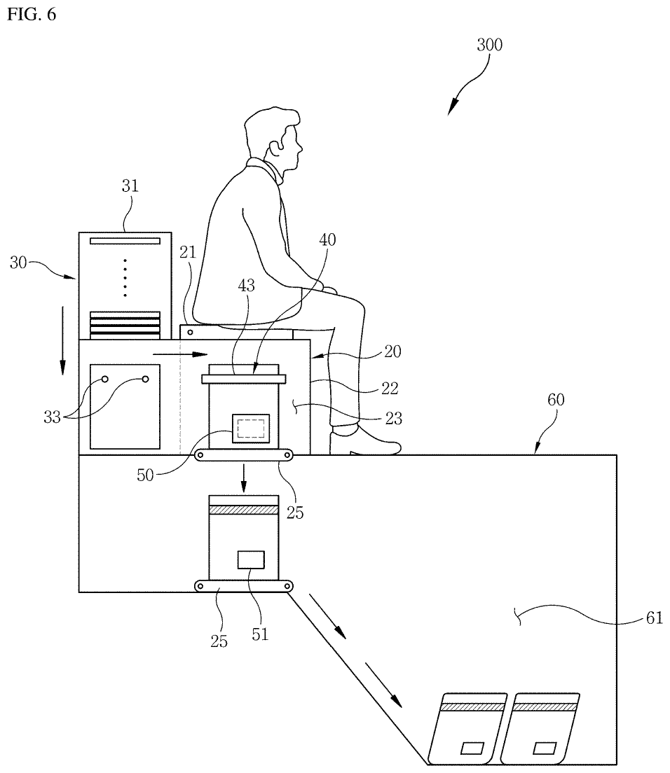

A case where the collection machine 60 includes the transfer member 63 is described above, but the transfer member 63 may not be included. For example, the transfer machine 25 residing in the sealing chamber 41 may be directly connected to the collection box 61 without the transfer member 63. In this case, the sealed plastic bag may be directly put into the collection box 61 by the linear rotational motion of the transfer machine 25. Alternatively, when the transfer machine 25 is implemented in the form of an open/closer, the transfer machine 25 may open the outlet of the inner space 23 such that the sealed plastic bag loaded on the transfer machine 25 will be directly dropped into the collection box 61. In order to prevent the sealed plastic bag from bursting in the process of falling into the collection box 61, the surface of the collection box 61 facing the transfer machine 25 may be formed as an inclined surface, as shown in FIG. 6.

As described above, the feces collecting and packaging apparatus 100 according to the present disclosure does not require human involvement by automating the entire process of collecting and packaging excrements. Therefore, it is possible to reduce the risk of infection that may occur in the process of collecting the entire feces. Further, the speed of collecting feces can be increased, and visual, psychological, and odor-related discomfort in stool collection process can be reduced.

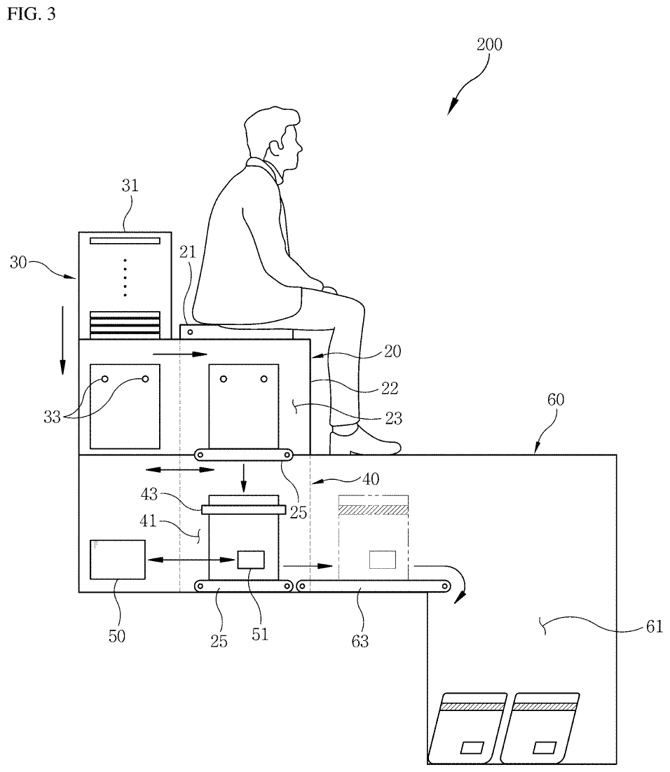

Meanwhile, the feces collecting and packaging apparatus 100 according to the present disclosure may be implemented as in first and second embodiments to be described hereinafter and shown in FIGS. 3 to 10. However, these embodiments are for illustrative purposes only, and the present disclosure is not limited thereto.

### First Embodiment

FIG. 3 is a diagram illustrating a feces collecting and packaging apparatus 200 according to a first embodiment of the present disclosure.

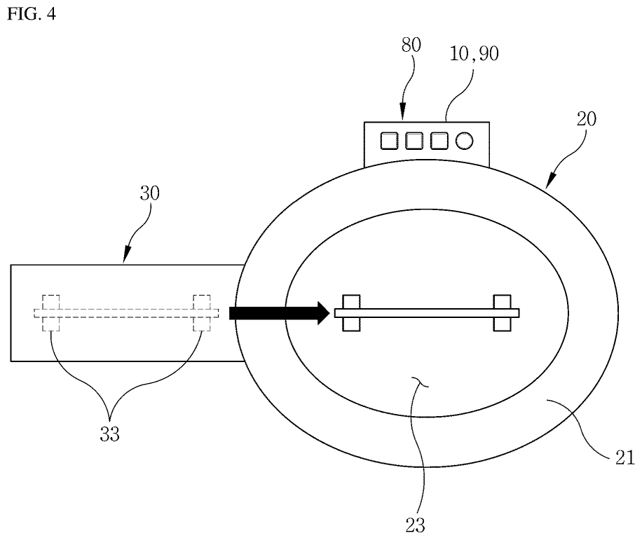

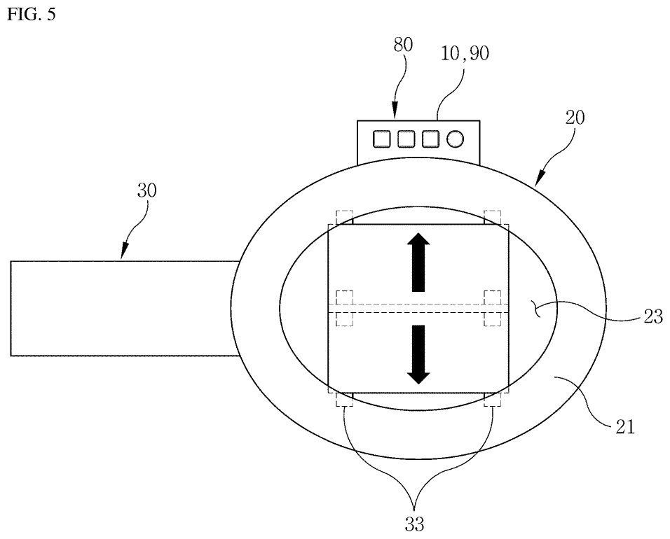

Referring to FIG. 3, the feces collecting and packaging apparatus 200 according to the first embodiment has a structure in which the bag supply machine 30 is installed at the rear of the main body 20, the sealing machine 40 is disposed under the main body 20, and the labeling machine 50 and the collection machine 60 are disposed on both sides of the sealing machine 40. A control box (80 in FIGS. 4 and 5) is provided on one side of the main body 20. The control box 80 includes the input device 10 and the controller 90.

Now, the feces collecting and packaging process performed by the feces collecting and packaging apparatus 200 according to the first embodiment will be described with reference to FIGS. 3 to 5. FIGS. 4 and 5 are diagrams illustrating a process of the apparatus shown in FIG. 3 in which the bag supply machine 30 dispenses a plastic bag to the inner space 23 of the main body 20.

An examinee is seated on the seat 21 of the main body 20 and then instructs the control box 80 to execute the feces collecting and packaging process. Alternatively or additionally, when a certain period of time elapses after the examinee is seated on the seat 21 of the main body 20, the control box 80 may automatically start the execution of the feces collecting and packaging process.

The examinee may put in information needed in fecal collection through the control box 80.

The control box 80 may output a guide message via text, voice or light output, so that the examinee can easily follow through the feces collecting and packaging process.

When an execution instruction is inputted through the control box 80, the gripper 33 grips a plastic bag from the bag loading box 31 of the bag supply machine 30, transfers it into the inner space 23 of the main body 20, and expands an upper opening of the plastic bag for receiving examinee's feces as shown in FIGS. 4 and 5. At this time, the plastic bag supplied into the inner space 23 is located under the seat 21 and loaded on the transfer machine 25 so as to stably receive feces from the examinee.

The bag supply machine 30 includes the bag loading box 31 and the gripper 33 installed behind the main body 20. Although FIG. 3 shows a case where the gripper 33 is disposed under the bag loading box 31 to receive a plastic bag from the bag loading box 31 in a vertical direction, the disclosure is not limited thereto. Alternatively, the bag loading box 31 and the gripper 33 may be arranged horizontally. In this case, the gripper 33 may pick up a plastic bag from the bag loading box 31 in a horizontal direction and then transfer the plastic bag into the inner space 23.

When the entire feces are completely contained in the plastic bag, the transfer machine 25 transfers the plastic bag containing the entire feces to the sealing chamber 41 located below the inner space 23. At this time, the gripper 33 releases a state of fixing (e.g., vacuum-adsorbing) the plastic bag so that the transfer machine 25 can transfer the plastic bag to the sealing chamber 41.

The transfer machine 25 moved to the sealing chamber 41 is located on the same line as the transfer member 63 of the collection machine 60.

Whether the feces are completely contained in the plastic bag may be determined depending on whether the examinee inputs a completion signal through the control box 80. Alternatively, when the examinee stands up from the seat 21, the controller 90 may determine that the feces are completely contained.

Next, in the sealing chamber 41, the sealing machine 43 seals the upper opening of the plastic bag containing the entire feces.

In addition, the labeling machine 50 attaches a label or sticker 51 recording examinee's feces collection information to the plastic bag in the sealing chamber 41.

Next, the transfer machine 25 (e.g., a belt conveyor) is linearly rotated in place (i.e., based on a closed loop of belt) toward the transfer member 63 (e.g., a belt conveyor) of the collection machine 60. Accordingly, the sealed plastic bag is transferred from the transfer machine 25 to the transfer member 63.

After transferring the sealed plastic bag containing the entire feces to the transfer member 63, the transfer machine 25 rises and returns to its original position. That is, the transfer machine 25 again blocks the outlet of the inner space 23.

In addition, the transfer member 63 transfers the sealed plastic bag containing the entire feces to the collection box 61, and the feces collecting and packaging process is completed.

### Second Embodiment

FIG. 6 is a diagram illustrating a feces collecting and packaging apparatus 300 according to a second embodiment of the present disclosure.

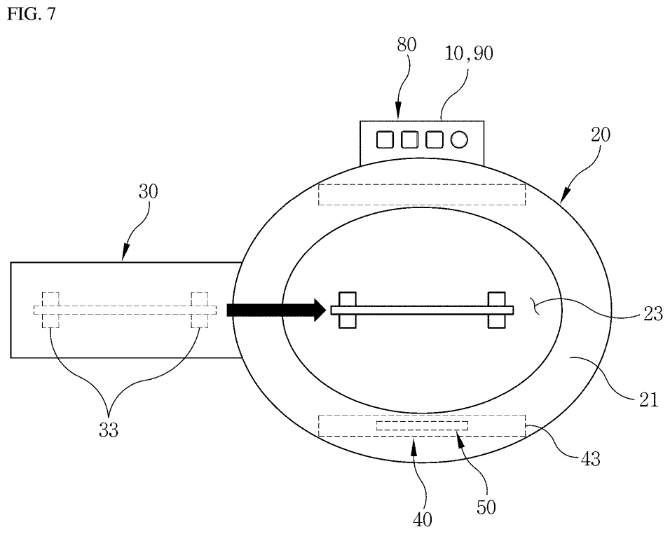

Referring to FIG. 6, the feces collecting and packaging apparatus 300 according to the second embodiment has a structure in which the bag supply machine 30 is installed at the rear of the main body 20, both the sealing machine 40 and the labeling machine 50 are installed in the inner space 23 of the main body 20, and the collection machine 60 is disposed under the main body 20. The control box (80 in FIGS. 7 to 10) is provided on one side of the main body 20. The control box 80 includes the input device 10 and the controller 90.

In the second embodiment, all of feces collection, sealing, and labeling are performed in the inner space 23 of the main body 20.

The collection machine 60 according to the second embodiment includes only the collection box 61 and does not include the transfer member 63 described above in the first embodiment. In this case, the transfer machine 25 performs the function of the transfer member 63 instead. However, it is also possible for the collection machine 60 according to the second embodiment to include the transfer member 63 as in the first embodiment.

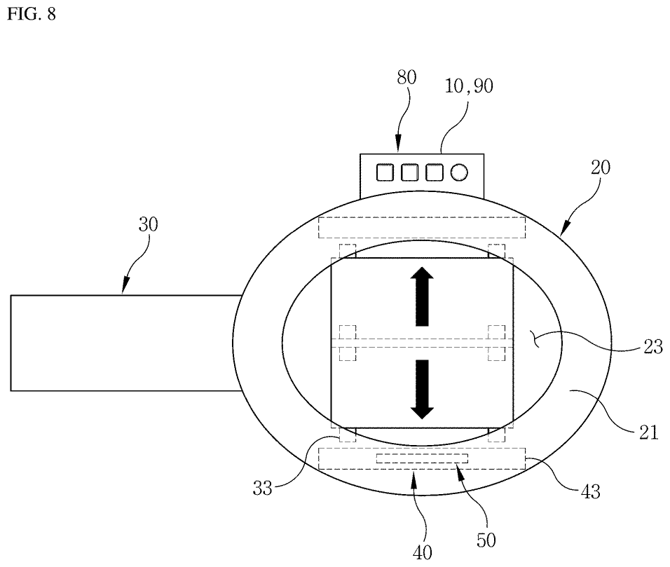

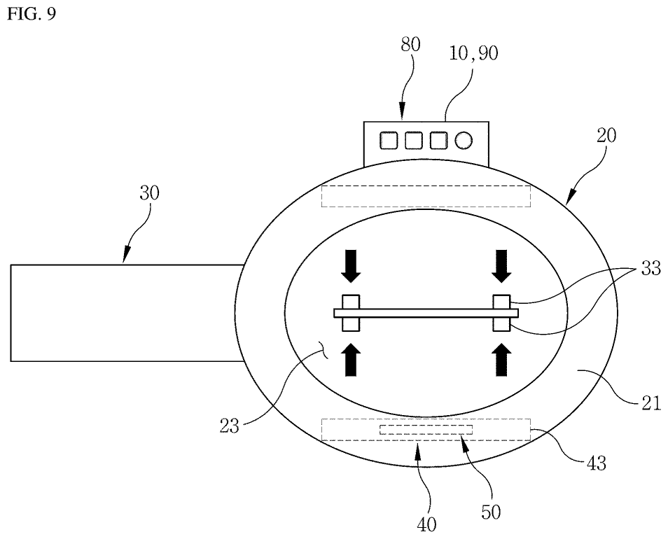

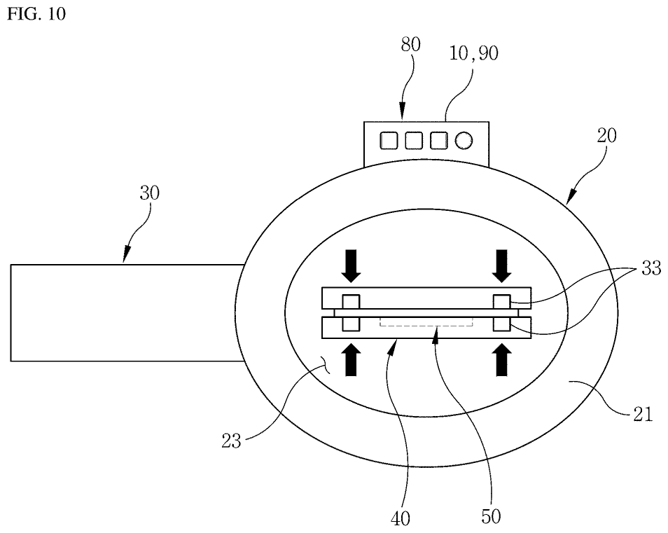

Now, the feces collecting and packaging process performed by the feces collecting and packaging apparatus 300 according to the second embodiment will be described with reference to FIGS. 6 to 10. FIGS. 7 and 8 are diagrams illustrating a process performed by the apparatus shown in FIG. 6 in which the bag supply machine 30 dispenses a plastic bag to the inner space 23 of the main body 20. FIGS. 9 and 10 are diagrams illustrating a process of the apparatus shown in FIG. 6 in which the sealing machine 40 seals the plastic bag and the labeling machine 50 attaches a label to the plastic bag.

An examinee is seated on the seat 21 of the main body 20 and then instructs the control box 80 to execute the feces collecting and packaging process. Alternatively or additionally, when a certain period of time elapses after the examinee is seated on the seat 21 of the main body 20, the control box 80 may automatically start the execution of the feces collecting and packaging process.

The examinee may input his/her feces collection information through the control box 80.

The control box 80 may output a guide message in text or voice or guide through light output so that the examinee can easily follow the feces collecting and packaging process.

When an execution instruction is inputted through the control box 80, the gripper 33 grips a plastic bag from the bag loading box 31 of the bag supply machine 30, transfers it into the inner space 23 of the main body 20, and expands an upper opening of the plastic bag for receiving examinee's feces as shown in FIGS. 7 and 8. At this time, the plastic bag supplied into the inner space 23 is located under the seat 21 and loaded on the transfer machine 25 so as to stably receive feces from the examinee.

The bag supply machine 30 includes the bag loading box 31 and the gripper 33 installed behind the main body 20. Although FIG. 3 shows a case where the gripper 33 is disposed under the bag loading box 31 to receive a plastic bag from the bag loading box 31 in a vertical direction, the disclosure is not limited thereto. Alternatively, the bag loading box 31 and the gripper 33 may be arranged horizontally. In this case, the gripper 33 may pick up a plastic bag from the bag loading box 31 in a horizontal direction and then transfer the plastic bag into the inner space 23.

When the entire feces are completely contained in the plastic bag, the sealing machine 43 residing in the inner space 23 seals the upper opening of the plastic bag containing the entire feces. That is, when the entire feces are completely enclosed in the plastic bag, the gripper 33 closes the upper opening of the plastic bag and then the sealing machine 43 seals the upper opening of the plastic bag.

In addition, the labeling machine 50 attaches the label or sticker 51 recording examinee's feces collection information to the plastic bag.

Then, the transfer machine 25 transfers the sealed plastic bag containing the entire feces to the collection machine 60 located below the inner space 23. At this time, the gripper 33 releases a state of fixing (e.g., vacuum-adsorbing) the plastic bag so that the transfer machine 25 can transfer the plastic bag to the collection machine 60.

Whether the feces are completely enclosed in the plastic bag may be determined depending on whether the examinee inputs a completion signal through the control box 80. Alternatively, when the examinee stands up from the seat 21, the controller 90 may determine that the feces are completely contained.

Next, the transfer machine 25 (e.g., a belt conveyor) is linearly rotated in place (i.e., based on a closed loop of belt) so as to transfer the sealed plastic bag containing the entire feces to the collection box 61. As a result, the feces collecting and packaging process is completed.

Meanwhile, the transfer machine 25 rises and returns to its original position. That is, the transfer machine 25 again blocks the outlet of the inner space 23.

While the present disclosure has been particularly shown and described with reference to an exemplary embodiment thereof, it will be understood by those skilled in the art that various changes in form and details may be made therein without departing from the scope of the present disclosure as defined by the appended claims.

What is claimed is:

1. An apparatus for collecting and packaging feces, the apparatus comprising:
a main body including a seat, a body block supporting the seat and having an inner space into which examinee's feces are discharged, and a transfer machine, installed at an outlet of the inner space opposite to an inlet connected to the seat, configured to transfer a plastic bag containing entire feces from the inner space;
a bag supply machine configured to dispense the plastic bag having an upper opening into the inner space;
a sealing machine installed under the transfer machine and sealing the upper opening of the plastic bag containing the entire feces;
a collection machine securing the sealed plastic bag from the inner space; and
a controller configured to direct the bag supply machine to supply the plastic bag into the inner space when the examinee is seated on the seat, direct the sealing machine to seal the upper opening of the plastic bag when the entire feces are completely contained, and control the collection machine to secure the sealed plastic bag from the inner space,
the controller further configured to:
instruct the transfer machine to close the outlet of the inner space while the plastic bag is supplied into the inner space and then the feces are contained in the plastic bag, and
in response to the entire feces being completely contained in the plastic bag, instruct the transfer machine to transfer the plastic bag under its own weight from the inner space to the sealing machine.

2. The apparatus of claim 1, wherein the bag supply machine includes:
a bag loading box loading a plurality of plastic bags therein; and
a gripper movably connected to the inner space, configured to grip the plastic bag one by one from the bag loading box, transfer the gripped plastic bag into the inner space, and expand or close the upper opening of the plastic bag.

3. The apparatus of claim 2, wherein in response to the entire feces being completely contained in the plastic bag, the controller is further configured to control the gripper to release a state of fixing the plastic bag such that the plastic bag containing the entire feces moves under its own weight from the inner space to the sealing machine.

4. The apparatus of claim 3, wherein the sealing machine includes:
a sealing chamber connected to the inner space via the transfer machine; and
a sealer automatically sealing the upper opening of the plastic bag moved into the sealing chamber.

5. The apparatus of claim 4, wherein the collection machine includes:
a collection box installed adjacent to the sealing chamber; and
a transfer member configured to transfer the sealed plastic bag from the sealing chamber to the collection box under control of the controller.

6. The apparatus of claim 1, further comprising:
a labeling machine installed in one of the sealing chamber, the inner space, or the bag supply machine, and attaching a label with examinee's feces collection information to the plastic bag under control of the controller.

7. An apparatus for collecting and packaging feces, the apparatus comprising:
a main body including a seat, a body block supporting the seat and having an inner space into which examinee's feces are discharged, and a transfer machine, installed at an outlet of the inner space opposite to an inlet connected to the seat, configured to transfer a plastic bag containing entire feces from the inner space;
a bag supply machine configured to dispense the plastic bag having an upper opening into the inner space;
a sealing machine installed in the inner space and sealing the upper opening of the plastic bag containing the entire feces;
a collection machine installed under the transfer machine and securing the sealed plastic bag from the inner space; and
a controller configured to direct the bag supply machine to dispense the plastic bag into the inner space when the examinee is seated, direct the sealing machine to seal the upper opening of the plastic bag when the entire feces are completely contained, and instruct the collection machine to secure the sealed plastic bag from the inner space,
the controller further configured to:
instruct the transfer machine to close the outlet of the inner space while the plastic bag is supplied into the inner space and then the feces are contained in the plastic bag, and
in response to the entire feces being completely contained in the plastic bag, instruct the transfer machine to transfer the sealed plastic bag under its own weight from the inner space to the collection machine.

8. The apparatus of claim 7, further comprising:
a labeling machine installed in one of the sealing chamber, the inner space, or the bag supply machine, and attaching a label with examinee's feces collection information to the plastic bag under control of the controller.
